(19) United States
(12) Patent Application Publication
(10) Pub. No.: US 2014/0286922 A1
(43) Pub. Date: Sep. 25, 2014

(54) Method of Measuring the Metabolic Indicators of Hair Follicles

(71) Applicant: The Procter & Gamble Company, Cincinnati, OH (US)

(72) Inventors: Ben Charlton HULETTE, CIncinnati, OH (US); Thomas Larry DAWSON, JR., Singapore (SG)

(73) Assignee: The Procter & Gamble Company, Cincinnati, OH (US)

(21) Appl. No.: 14/219,214

(22) Filed: Mar. 19, 2014

Related U.S. Application Data
(60) Provisional application No. 61/803,260, filed on Mar. 19, 2013.

(51) Int. Cl. G01N 33/50

(52) U.S. Cl. CPC G01N 33/5088
USPC 424/94.1; 514/556; 435/14; 514/690; 514/263.34; 514/565; 514/440; 514/355; 514/529; 514/311; 514/20.7; 514/578; 514/47

(57) ABSTRACT

A method of measuring the effect of an active agent or a stressor on the baseline metabolic indicators of one or more hair follicles, the method including (a) obtaining one or more hair follicles; (b) placing the one or more hair follicles in a vessel, wherein the one or more hair follicles are positioned under one or more sensors; (c) using the one or more sensors to measure the one or more hair follicles' baseline metabolic indicators for both glycolysis and oxidative phosphorylation; (d) exposing the one or more hair follicles to an active agent or a stressor; and (e) using the one or more sensors to measure the one or more hair follicles' respondent metabolic indicators for both glycolysis and oxidative phosphorylation.

### FIELD OF THE INVENTION

The present invention is directed, generally, to a method of measuring the metabolic indicators of one or more hair follicles. More specifically, the present invention is directed to a method of measuring the effect of an active agent or a stressor on the baseline metabolic indicators of one or more hair follicles.

### BACKGROUND OF THE INVENTION

A fundamental basis for life is the need and ability of an organism to generate energy. The metabolic pathways of the cells that convert the useful components of food (e.g., carbohydrates, fats, and proteins) into usable energy are complex and may be affected by a variety of factors in ways that are not completely understood. Hair follicles are no exception.

Hair follicles are among the most metabolically active cells in the body, utilizing approximately 670 kcal to generate a single gram of hair fiber. This is evidenced by the loss of hair follicle function whenever there is a perturbation of energy production in the hair follicle. This occurs in chemotherapy, where drugs are specifically targeted to attack rapidly proliferating and energy intensive cells. This is also evidenced by hair loss being among the first symptoms of multiple physiologic changes that affect energy production in the body including stress or a sudden change in diet.

Hair follicles include a variety of different cell types that function together in a dynamic, complex relationship to maintain the health of the hair. For example, keratinocytes proliferate and differentiate to create the hair shaft, melanocytes provide melanin for hair pigmentation, fibroblasts synthesize the extracellular matrix and collagen, and dermal papilla cells regulate the complex interactions of the above cells to maintain hair health. Similarly, other cells found in or around the hair follicle such as myocytes, stem cells, sebocytes, neurocytes, and adipocytes all require energy derived from complex metabolic pathways which can be impacted by a variety of different factors.

There is a growing awareness of the impact of various stressors on cellular bioenergetics, cell aging, and disease (e.g., cancer, neurodegenerative diseases, diabetes, and cardiovascular disorders). One of the theories underlying the impact of various stressors is the Free Radical Theory. Under the Free Radical Theory, exposure of mammalian cells to reactive oxygen species (“ROS”) causes damage to cellular structures and organelles such as the mitochondria. ROS are highly reactive molecules that contain oxygen. ROS are formed within cells as a natural byproduct of the normal metabolism of oxygen and play a role in cell signaling and homeostasis. However, when a cell is exposed to a stressor such as heat, harsh chemicals, or UV radiation, ROS levels can increase, and in some instances, dramatically.

Hair follicle cells defend against ROS by using redox regulators such as glutathione and nicotinamide adenine dinucleotide as well as various enzymes that can neutralize ROS. However, these defenses can be overwhelmed by the elevated spike from stressor-induced ROS, leading to both acute and chronic alterations in metabolism efficiencies, causing overall follicular dysfunction. To complicate matters, the variety of cells types associated with human hair follicles and the complexity of their metabolic pathways makes it difficult to identify suitable compounds to help combat the hair amount diminution effect associated with exposure to particular oxidative stressors. Certain oxidative stressors affect different types of cells and metabolic pathways differently. This makes it difficult to select suitable benefit agents that combat the undesirable affects of a particular stressor or stressors.

Accordingly, there is a need for a method of measuring the effect of active agents and/or stressors on the metabolic indicators of hair follicles. There is also a need to identify benefit agents which can be used in hair care compositions using the foregoing method.

### SUMMARY OF THE INVENTION

According to an embodiment of the invention, there is provided a method of measuring the effect of an active agent or stressor on the baseline metabolic indicators of one or more hair follicles, the method comprising (a) obtaining one or more hair follicles; (b) placing the one or more hair follicles in a vessel, wherein the one or more hair follicles are positioned under one or more sensors; (c) using the one or more sensors to measure the one or more hair follicles' baseline metabolic indicators for both glycolysis and oxidative phosphorylation; (d) exposing the one or more hair follicles to an active agent or a stressor; and (e) using the one or more sensors to measure the one or more hair follicles' respondent metabolic indicators for both glycolysis and oxidative phosphorylation.

According to another embodiment, the active agent may be identified as a benefit agent when one of the respondent metabolic indicators corresponds to an improvement in the one or more hair follicles' metabolic activity when compared to the corresponding baseline metabolic indicators.

According to yet another embodiment, there is provided a hair care composition comprising the active agent as identified above.

### DETAILED DESCRIPTION OF THE INVENTION

All percentages are by weight of the hair-care composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements.

“Benefit agent,” as used herein, means a compound or combination of compounds that, when applied to hair and/or scalp, provides an acute and/or chronic benefit to a type of cell commonly found therein. The benefit agent may achieve a benefit by regulating and/or improving the metabolic indicators of the hair and/or scalp or its associated cells.

“Dermatologically acceptable carrier,” as used herein, means that the carrier is suitable for use in compositions which come in contact with human hair and/or skin without undue toxicity, incompatibility, instability, allergic response, and the like.

“Hair care composition,” as used herein, means any composition capable of providing a desired visual or tactile effect on an area of the hair and/or scalp of an animal or human. The visual or tactile effect may be temporary, semi-permanent, or permanent. Some non-limiting examples of hair care compositions include products that clean the hair, such as shampoos. Other products may include conditioners, leave-on treatments, styling aids, hair colorants, and the like.

“Hair follicle,” as used herein, means an intact mammalian hair follicle and/or a three-dimensional tissue-engineered hair follicle. Mammalian hair follicles may include pet hair follicles.

“Non-lethally,” as used herein, means that a test procedure or measurement does not kill or destroy the hair follicle cells being tested or observed. For example, non-lethally detecting a baseline metabolic indicator means that at least 75% of the hair follicle cells are viable after the detection (e.g., 80%, 85%, 90%, 95% or even up to 99% or more of the cells remain viable). Ideally, 100% of the cells are viable after a non-lethal test, but it is to be appreciated that the death or destruction of some cells may be unavoidable and/or unrelated to the test.

“Stressor,” as used herein, means any element that causes the formation of undesirable reactive oxygen species in a cell. Non-limiting examples of stressors may be selected from the group consisting of hair dyes, hair sprays, oxidizing agents, solvents, heat, hot oils, perm treatments, physical perturbation, ultraviolet radiation, cigarette smoke, ozone, engine exhaust, diesel exhaust, smog, surfactants, radiation, and combinations thereof

Provided is a method of measuring the effect of an active agent on the baseline metabolic indicators of one or more hair follicles may comprising (a) obtaining one or more hair follicles; (b) placing the one or more hair follicles in a vessel, wherein the one or more hair follicles are positioned under one or more sensors; (c) using the one or more sensors to measure the one or more hair follicles' baseline metabolic indicators for both glycolysis and oxidative phosphorylation; (d) exposing the one or more hair follicles to an active agent or a stressor; and (e) using the one or more sensors to measure the one or more hair follicles' respondent metabolic indicators for both glycolysis and oxidative phosphorylation.

Two key metabolic pathways for mammalian cells to produce energy are the oxidative phosphorylation (“oxphos”) pathway and the glycolysis pathway. Both pathways are necessary to maintain a healthy energy balance within most mammalian hair follicles. Oxidative phosphorylation involves the transfer of electrons from electron donors to electron acceptors such as oxygen in redox reactions. These redox reactions release energy that is used to form ATP. In mammals, the redox reactions are carried out by a series of protein complexes within the mitochondria membrane, and the linked sets of proteins are called electron transport chains. The energy released by electrons flowing through this electron transport chain is used to transport protons across the mitochondrial membrane, in a process called chemiosmosis, which generates potential energy in the form of a pH gradient and an electrical potential across this membrane. An enzyme commonly known as ATP synthase allows the potential energy to be used to generate ATP. Because the oxidative phosphorylation pathway uses oxygen to generate ATP, the rate at which a hair follicle consumes oxygen may be used as a metabolic indicator of the hair follicle. That is, the oxygen consumption rate of the hair follicle may be directly correlated to energy production by the hair follicle via oxidative phosphorylation. Additionally or alternatively, the carbon dioxide production rate may also be used as a metabolic indicator, since carbon dioxide is a by-product of cellular metabolism. A higher oxygen consumption rate or carbon dioxide production rate may indicate an increase in energy production from the oxidative phosphorylation pathway, and thus an improvement in the metabolism and/or health of a hair follicle. Conversely, a lower oxygen consumption rate or carbon dioxide production rate may indicate a decrease in the metabolism and/or health of the hair follicle.

Glycolysis is the metabolic pathway that converts glucose into pyruvate. The free energy released in this process is used to form ATP and NADH (reduced NAD). Glycolysis is a definite sequence of ten reactions involving ten intermediate compounds (one of the steps involves two intermediates) that typically occurs in the cytosol of hair follicle cells. The intermediates provide entry points to glycolysis. For example, most monosaccharides such as fructose, glucose, and galactose, can be converted to one of these intermediates. The intermediates may also be directly useful. For example, the intermediate dihydroxyacetone phosphate (DHAP) is a source of the glycerol that combines with fatty acids to form fat. A by-product of glycolysis is lactic acid, which can form a lactate anion plus a proton in solution. Thus, lactic acid, lactate or proton concentration can be used as a metabolic indicator of glycolysis. That is, a change in extracellular pH or extracellular acidification rate may be directly correlated to energy production by the cell via the glycolysis pathway. A higher extracellular acidification rate may indicate an increase in energy production via the glycolysis pathway, and thus an improvement in the metabolism and/or health of a hair follicle. Conversely, a lower extracellular acidification rate may indicate a decrease in glycolysis metabolism and/or health of a hair follicle.

Provided is a method of measuring the effect of an active agent or a stressor on the baseline metabolic indicators of one or more hair follicles, the method comprising (a) obtaining one or more hair follicles; (b) placing the one or more hair follicles in a vessel, wherein the one or more hair follicles are positioned under one or more sensors; (c) using the one or more sensors to measure the one or more hair follicles' baseline metabolic indicators for both glycolysis and oxidative phosphorylation; (d) exposing the one or more hair follicles to an active agent or a stressor; and (e) using the one or more sensors to measure the one or more hair follicles' respondent metabolic indicators for both glycolysis and oxidative phosphorylation.

The method of measuring the effect of an active agent or a stressor on the baseline metabolic indicators of one or more hair follicles may comprise obtaining one or more hair follicles. The one or more hair follicles may be obtained from any mammal including pets and humans. In an embodiment, the one or more hair follicles may be from cow skin. In another embodiment, the one or more hair follicles may comprise one or more three-dimensional tissue-engineered hair follicles.

The method of measuring the effect of an active agent or a stressor on the baseline metabolic indicators of one or more hair follicles may include screening for one or more hair follicles that are growing.

Screening for growing hair follicles may be accomplished by the following method:
(1) isolating one or more hair follicles from mammalian tissue under a dissecting microscope using a scalpel and fine-tipped forceps without damaging the bulb region or the dermal sheath, transecting below the sebaceous gland;
(2) placing the hair follicles in a Williams Medium E supplemented with 2 mM L-glutamine, insulin, hydrocortisone, and antibiotic/antimycotic;
(3) placing each hair follicle in a well of a culture plate containing 0.5 mL of supplemented Williams Medium E;
(4) obtaining digital baseline images with a Moticam 2300 camera connected to an Olympus SZH Micro-Dissecting Scope under 2.5× magnification with an Olympus DF Plan 1× Optic;
(5) placing the culture plate in a humidified incubator at 37° C. with 5% CO2 for 24 hours; and
(6) comparing the hair follicles to the baseline images—growth is determined when simultaneous root sheath and fiber extension occur.

The method of measuring the effect of an active agent or a stressor on the baseline metabolic indicators of one or more hair follicles may comprise placing the one or more hair follicles in a vessel, wherein the one or more hair follicles are positioned under one or more sensors. The one or more sensors may be sensors located on any suitable device for measuring metabolic indicators, including the XF Extracellular Flux Analyzer available from Seahorse Bioscience, Massachusetts. In an embodiment, the vessel may be an islet vessel or a multi-well microplate.

In an embodiment, the one or more hair follicles may be secured to the vessel using a cellular adhesive. Any suitable cellular adhesive may be used, including Cell-Tak™ (BD Biosciences, Bedford, Mass.). In an embodiment, the cellular adhesive may not be mixed with a neutralizing buffer prior to securing the one or more hair follicles to the vessel.

Cellular adhesive may be applied to the vessel using the following method:
(1) adding 20 microliters (μl) of neat Cell-Tak™ (BD Biosciences, Bedford, Mass.) to the vessel;
(2) allowing the vessel to air dry in a laminar flow containment cabinet;
(3) rinsing the vessel once with 1 ml of aqueous 70% ethanol;
(4) rinsing the vessel twice with 1 ml sterile water; and
(5) allowing the vessel to air dry.

The method of measuring the effect of an active agent or a stressor on the baseline metabolic indicators of one or more hair follicles may include using the one or more sensors to measure the one or more hair follicles' baseline metabolic indicators for both glycolysis and oxidative phosphorylation. In an embodiment, the baseline metabolic indicators are detected simultaneously. In an embodiment, the one or more hair follicle's baseline metabolic indicators may be measured non-lethally. In an embodiment, the measurements of the baseline metabolic indicators may be taken in a controlled environment.

The method of measuring the effect of an active agent or a stressor on the baseline metabolic indicators of one or more hair follicles may include exposing the one or more hair follicles to an active agent or a stressor. Non-limiting examples of an active agent may be selected from the group consisting of caffeine, carnitine, creatine, alpha lipoic acid, niacinamide, coenzyme Q10, acyl carnitine, acetyle carnitine, ubiquinone, ubiquinone derivatives, ethoxyquin, wound healing peptides, wound healing peptides coupled to acyl groups (i.e. palmitic acid), taurine, acetyl coenzyme A, and combinations thereof. In an embodiment, the active agent may be niacinamide. Non-limiting examples of the stressor may be selected from the group consisting of hair dyes, hair sprays, solvents, heat, hot oils, perm treatments, physical perturbation, ultraviolet radiation, cigarette smoke, ozone, engine exhaust, diesel exhaust, smog, surfactants, radiation, and combinations thereof. In an embodiment, the method comprises exposing the one or more hair follicles to both an active agent and a stressor, in any order.

The method of measuring the effect of an active agent or a stressor on the baseline metabolic indicators of one or more hair follicles may include using the one or more sensors to measure the one or more hair follicle's respondent metabolic indicators for both glycolysis and oxidative phosphorylation. In an embodiment, the respondent metabolic indicators may be detected simultaneously. In an embodiment, the measurements of the respondent metabolic indicators may be taken in a controlled environment.

In an embodiment, the baseline metabolic indicator and the respondent metabolic indicator for oxidative phosphorylation is an oxygen consumption rate. In another embodiment, the baseline metabolic indicator and the respondent metabolic indicator for oxidative phosphorylation is a carbon dioxide generation rate.

In an embodiment, the baseline metabolic indicator and the respondent metabolic indicator for the glycolysis pathway is an extracellular acidification rate.

The method of measuring the effect of an active agent or a stressor on the baseline metabolic indicators of one or more hair follicles may include exposing the hair follicle to a stressor at any time before measuring the hair follicle's respondent metabolic indicators.

The provided method enables the non-lethal, simultaneous detection of metabolic indicators associated with the oxidative phosphorylation and glycolysis metabolic pathways in a controlled environment. The method also enables the collection of kinetic data. When assessing the metabolic response to a stressor or active agent, it is important to assess both metabolic pathways simultaneously to understand how the two metabolic pathways interact with the stressor or active agent and/or to one another. Additionally, it is important to monitor the metabolic pathways in real time (i.e., repeating periodic measurements) to observe trends and/or transient responses that may be missed when using methods that provide only static data. However, in some embodiments, destructive tests may be suitable herein.

It is also important to detect the metabolic indicator in a controlled environment to reduce the likelihood of artifact introduction. A suitable controlled environment should minimize or prevent any undesirable influence by external environmental conditions (e.g., temperature, pressure, humidity, light, contact by undesirable gaseous, liquid or solid contaminants). For example, if the metabolic indicator being detected is oxygen consumption rate and the test sample is open to the environment, the measured oxygen concentration may not accurately reflect the amount of oxygen consumed by the hair follicle cells, since at least some of the consumed oxygen may be replaced by environmental oxygen. Additionally, the test method itself should not introduce artifact into the measurement.

It is to be appreciated that environmental changes such as a change in the temperature of the medium may result in unwanted measurement errors. In particular, the capacity of the media to hold dissolved gasses changes with temperature, and therefore may result in an apparent change in dissolved gas concentration as the media seeks equilibrium with the surrounding environment. Further, the measurement properties of at least some types of sensors may change with temperature. Accordingly, it may be particularly desirable to control the environmental conditions such as the temperature of the medium in the test vessel and/or surrounding environment or apply a correction factor to the measurement. Similarly, any evaporation from the media due to other uncontrolled environmental conditions such as humidity or exposure to air currents may artificially impact the measurements made from various sensors including those of dissolved gases, ions, and temperature. Thus, providing a controlled environment to minimize or eliminate these factors is important.

In some embodiments, the device used to detect the metabolic parameters may include a stage adapted to receive the test vessels (e.g., multi-well microplate) holding the hair follicle. The device may also include a plunger configured to receive a barrier for isolating the environment within the test vessel from the external environment. The barrier may be configured to cooperate with a portion of the test vessel to seal the opening in the test vessel, for example, by mating with a seat or step in the test vessel wall. Additionally or alternatively, the plunger and barrier may be configured to reduce and/or expand the volume of the test vessel and the volume of the medium within the test vessel including at least a portion of the hair follicle (e.g., 5-50%). For example, the barrier may be inserted into and/or retracted out of the test vessel by relative movement of the stage and the plunger. In some embodiments, the method may include perfusing additional media through the vessel and/or replenishing the medium. Reducing the volume of the medium enables the temporary creation of a highly concentrated volume of cells within a larger volume of cell media to improve the ability of the sensor(s) to detect sensitive changes in metabolic indicators in the medium that result from biological activity of the hair follicle cells. By temporarily, rather than permanently, reducing the media volume (and therefore concentrating the cell/media mixture), cells are exposed to a non-normal environment for only a brief period of time, and therefore may not be adversely affected by the measurement process.

The instrument should also include a sensor capable of analyzing the desired metabolic indicator(s). In some embodiments, the sensory may be disposed on the barrier and/or plunger. The sensor should be in sensing communication with the medium and configured to detect the desired metabolic indicator. The sensor may be configured to sense the presence and/or the concentration of the metabolic indicator; sense a rate of change of a concentration of the metabolic indicator; and/or sense a first concentration of a first metabolic indicator, sensing a second concentration of a second metabolic indicator, and determining a relationship between the first concentration and the second concentration. It may be desirable to configure the sensor to detect the metabolic parameter without disturbing the one or more hair follicles. The instrument may also include a computer programmed to automate one or more aspects of the tests, including data collection and recording, cycling through one or more test steps and transferring an active agent or stressor to the extracellular environment. In some embodiments, the sensor may be coupled to the computer.

Suitable nonlimiting examples of devices that provide a controlled environment; non-lethal and simultaneous detection of metabolic indicators; and kinetic data are disclosed in U.S. Pat. Nos. 7,276,351; 7,638,321; and 7,851,201, to Teich et al.; and U.S. Pat. No. 8,202,702 to Neilson, et al. A particularly suitable device is the XF Extracellular Flux Analyzer available from Seahorse Bioscience, Massachusetts.

Oxygen consumption rate and extracellular acidification rate may be detected using an XF Extracellular Flux Analyzer or equivalent. The device should be capable of non-lethally and simultaneously detecting metabolic indicators of the oxphos and glycolysis pathways in a controlled environment, as well as providing kinetic data. The one or more hair follicles may be provided in a multi-well plate suitable for use with the instrument (e.g., 24-well plate or 96-well plate) and washed prior to testing. The one or more hair follicles may be washed by any suitable means known in the art (e.g., using a Seahorse Biosciences XF prep station). When washing the one or more hair follicles, it may be desirable to remove the medium from the wells and wash the cells three times with a suitable amount of test medium (e.g., 180 μL in a 96-well plate or 600 μL in a 24-well plate). After washing the one or more hair follicles, a suitable amount of test medium is placed in each well, and the hair follicles are equilibrated at 37° C. in a CO₂-free incubator for 1-1.5 hours prior to placing the plate in the instrument for testing. Following the equilibration period, load the plate containing the one or more hair follicles into the instrument and equilibrate according to manufacturer's instructions. The entire test is conducted at 37° C. In some embodiments, it may be desirable to set the instrument to provide a three minute mix cycle, a two minute wait cycle, and a 3 minute measurement cycle for keratinocytes and a two minute mix cycle, two minute wait cycle and 3 minute measurement cycle for fibroblasts. The cycles should be repeated for at least 88 minutes. It is to be appreciated that the cycles and times may be modified as desired.

The method of measuring the effect of an active agent or a stressor on the baseline metabolic indicators of one or more hair follicles may include identifying the active agent as a benefit agent when one of the respondent metabolic indicators corresponds to an improvement in the one or more hair follicles' metabolic activity when compared to the corresponding baseline metabolic indicators. In an embodiment, a higher extracellular acidification rate may indicate an increase in energy production via the glycolysis pathway, and thus an improvement in the metabolism and/or health of the one or more hair follicles. In an embodiment, a higher oxygen consumption rate or carbon dioxide production rate may indicate an increase in energy production from the oxidative phosphorylation pathway, and thus an improvement in the metabolism and/or health of a hair follicle

In an embodiment, a hair care composition may comprise the benefit agent as identified above and a dermatologically acceptable carrier. Suitable hair care compositions herein may be in any one of a variety of forms known in the art, including, for example, an emulsion, liquid, solid, milk, cream, gel, mouse, ointment, paste, serum, stick, spray, tonic, aerosol, foam, and the like.

The hair care composition may also comprise one or more optional ingredients. Nonlimiting examples of optional ingredients suitable for use therein are described in U.S. Publication No. 2010/0239510 filed by Ha et al., on Jan. 21, 2010.

The hair care composition incorporating may be prepared according to conventional methods known in the art of making compositions and topical compositions. Such methods typically involve mixing of ingredients in or more steps to a relatively uniform state, with or without heating, cooling, application of vacuum, and the like. The hair care composition may be provided in a package sized to store a sufficient amount of the composition for a treatment period. The size, shape, and design of the package may vary widely.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Additionally, properties described herein may include one or more ranges of values. It is to be understood that these ranges include every value within the range, even though the individual values in the range may not be expressly disclosed.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of measuring the effect of an active agent or a stressor on the baseline metabolic indicators of one or more hair follicles, the method comprising, in any order:
a. obtaining one or more hair follicles;
b. placing the one or more hair follicles in a vessel, wherein the one or more hair follicles are positioned under one or more sensors;
c. using the one or more sensors to measure the one or more hair follicles' baseline metabolic indicators for both glycolysis and oxidative phosphorylation;
d. exposing the one or more hair follicles to an active agent or a stressor; and
e. using the one or more sensors to measure the one or more hair follicles' respondent metabolic indicators for both glycolysis and oxidative phosphorylation.

2. The method of claim 1, comprising, in any order:
a. exposing the one or more hair follicles to an active agent; and
b. exposing the one or more hair follicles to a stressor.

3. The method of claim 1, wherein the baseline metabolic indicators are detected simultaneously.

4. The method of claim 1, wherein the respondent metabolic indicators are detected simultaneously.

5. The method of claim 1, wherein the baseline metabolic indicator and the respondent metabolic indicator for oxidative phosphorylation is an oxygen consumption rate.

6. The method of claim 1, wherein the baseline metabolic indicator and the respondent metabolic indicator for oxidative phosphorylation is a carbon dioxide generation rate.

7. The method of claim 1, wherein the baseline metabolic indicator and the respondent metabolic indicator for the glycolysis pathway is an extracellular acidification rate.

8. The method of claim 1, wherein the one or more hair follicles comprise one or more cow hair follicles.

9. The method of claim 1, wherein the one or more hair follicles comprise one or more three-dimensional tissue-engineered hair follicles.

10. The method of claim 1, wherein the one or more hair follicle's baseline metabolic indicators are measured non-lethally.

11. The method of claim 1, wherein the vessel is an islet vessel or a multi-well microplate.

12. The method of claim 1, further comprising securing the one or more hair follicles in the vessel with a cellular adhesive.

13. The method of claim 12, wherein the cellular adhesive is not mixed with a neutralizing buffer prior to securing the one or more hair follicles in the vessel.

14. The method of claim 1, wherein the stressor is selected from the group consisting of hair dyes, hair sprays, oxidizing agents, solvents, heat, hot oils, perm treatments, physical perturbation, ultraviolet radiation, cigarette smoke, ozone, engine exhaust, diesel exhaust, smog, surfactants, radiation, and combinations thereof.

15. The method of claim 1, further comprising screening for one or more hair follicles that are growing.

16. The method of claim 1, wherein the measurements of the baseline metabolic indicators and respondent metabolic indicators are taken in a controlled environment.

17. The method of claim 1, wherein the active agent is selected from the group consisting of caffeine, carnitine, creatine, alpha lipoic acid, niacinamide, coenzyme Q10, acyl carnitine, acetyle carnitine, ubiquinone, ubiquinone derivatives, ethoxyquin, wound healing peptides, wound healing peptides coupled to acyl groups, taurine, acetyl coenzyme A, and combinations thereof.

18. The method of claim 17, wherein the active agent is carnitine.

19. The method of claim 1, further comprising identifying the active agent as a benefit agent when one of the respondent metabolic indicators corresponds to an improvement in the one or more hair follicles' metabolic activity when compared to the corresponding baseline metabolic indicators.

20. A hair care composition comprising:
a. the benefit agent identified by the method of claim 19; and
b. a dermatologically acceptable carrier.
